(19) United States
(12) Patent Application Publication
(10) Pub. No.: US 2023/0372623 A1
(43) Pub. Date: Nov. 23, 2023

(54) System And Device For Dispensing A Product

(71) Applicant: Nemera La Verpillière, LA VERPILLIERE (FR)

(72) Inventors: Marc Todesco, TIGNIEU-JAMEYZIEU (FR); Gaëtan Grenot, LYON (FR)

(21) Appl. No.: 18/030,350

(22) PCT Filed: Oct. 5, 2021
(86) PCT No.: PCT/EP2021/077474

§ 371 Date: Apr. 5, 2023

(30) Foreign Application Priority Data
Oct. 5, 2020 (FR) 2010167

(51) Int. Cl. A61M 5/24; A61M 5/142

(52) U.S. Cl. CPC A61M 5/2459; A61M 5/14244; A61M 2205/3306; A61M 2205/3331

(57) ABSTRACT

The invention relates to a product dispensing device, having a reservoir of product provided with a collar delimiting an opening, a membrane closing the opening and having an inner side oriented towards the reservoir and an outer side opposite to the inner side, a securing ring for securing the membrane to the collar, the membrane being bistable in that it is configured to switch from a first stable position, in which the membrane bulges out in a first direction such that it protrudes into the reservoir and closes the reservoir, into a second stable position, in which the membrane bulges out in a second direction opposite to the first direction so as to be pierced by a needle disposed on the outer side of the membrane.

### FIELD OF THE INVENTION

The invention relates to a system for dispensing a product, in particular a liquid product intended to be introduced in a site of a subject. In addition, the invention relates to a dispensing device comprising the dispensing system.

### BACKGROUND OF THE INVENTION

The state of the art already describes a dispensing device for dispensing medication comprising a reservoir and a membrane closing off the reservoir. To dispense product contained in the reservoir, the membrane is deformable from a storage position to an assembly position in which a needle bursts the membrane. Such a membrane is particularly thin, approximately 0.1 to 0.2 mm, and is therefore difficult to manufacture and assemble since this requires numerous precautions and very precise positioning of the membrane. In addition, it is difficult to achieve a deformation of the membrane performed in a reproductible manner, since it is very thin and must be subjected to a relatively high differential pressure to ensure that it deforms sufficiently to actually come into contact with the needle and burst.

The invention aims in particular to propose a dispensing device for dispensing a product that is easier to produce, while preserving the sterility of the product contained in the reservoir, until the product is dispensed out of the reservoir.

Thus, the invention relates in particular to a dispensing device for dispensing a product, comprising a reservoir of the product provided with a collar delimiting an opening, a membrane closing the opening and thus having an inner side oriented towards the reservoir and an outer side opposite to the inner side, a securing ring for securing the membrane to the collar, the membrane being bistable in that it is configured to switch from a first stable position, in which the membrane bulges out in a first direction such that it protrudes into the reservoir and closes the reservoir, into a second stable position, in which the membrane bulges out in a second direction opposite to the first direction so as to be pierced by a needle disposed on the outer side of the membrane.

Thus, it is proposed to use a bistable membrane which, when it changes from the first stable position to the second stable position, pass through an intermediate unstable position which generates stresses in the membrane and which push it towards the second stable position. Thus, the membrane “snaps” from the first stable position into the second stable position, which guarantees that the membrane is pierced by the needle. The reproducibility of the bistable membrane deformation is therefore increased since the bistable membrane is not necessarily thin, and its operation does not depend on an uncertain ability to be inflated. Furthermore, such a dispensing device is particularly simple to produce, since it only requires the use of a securing ring for securing the membrane to the collar, while preserving the sterility of the reservoir and the product contained in the reservoir, from the time the reservoir is assembled in the dispensing device until the product is dispensed out of the reservoir, as well as during and after filling the reservoir. The dispensing device is particularly simple to produce since, thanks to the securing ring for securing the membrane to the reservoir, the reservoir can be a standard reservoir such as those commonly used in the pharmaceutical field, without requiring any specific feature for securing the membrane. This securing ring can for example form a ferrule. Lastly, the membrane makes the assembly particularly simple since, preferably, its only function is to switch between a storage and assembly position corresponding to the first stable position and a pierced position corresponding to the second stable position, with no other additional function.

### SUMMARY OF THE INVENTION

According to other optional characteristics of the dispensing device taken alone or in combination:
The dispensing device comprises the needle that can pierce the membrane when the membrane is in the second stable position.
In its first stable position, the membrane closes the reservoir so as to prevent product from being dispensed.
In its second stable position, the membrane bulges out in the second direction such that it protrudes out of the reservoir.
The securing ring is made of thermoplastic material or metallic material, preferably aluminum. Thus, holding the membrane on the reservoir is particularly inexpensive.
The reservoir is made of glass or thermoplastic material. Thus, the reservoir is particularly simple and inexpensive to produce. In particular, it may be a standard glass reservoir, commonly used in the pharmaceutical field and simple to sterilize.
The reservoir has the shape of a syringe provided with a collar. Thus, the reservoir is particularly simple to produce. According to another variant, the reservoir has the shape of a cartridge provided with a collar. According to yet another variant, the reservoir consists of a flexible pouch connected to a collar that is more rigid than the flexible pouch.
The needle is a needle for connecting to a product distribution tubing, preferably a needle for connecting to a catheter for dispensing product. As an alternative, the needle is a needle for injecting product, which comprises one end for piercing the membrane and one opposite end for injecting product into the site of a subject.
The opening is closed only by the membrane. Thus, the dispensing device is particularly simple to produce.
The membrane is mounted directly or indirectly on the collar of the reservoir, preferably directly. In particular, it is crimped directly onto the collar of the reservoir by the securing ring. Thus, the dispensing device is particularly simple to produce.
The membrane bulges out in the first direction such that it protrudes into the collar of the reservoir.
The membrane has a peripheral part which is compressed axially between the securing ring and the collar of the reservoir. The axial direction is defined in reference to a central axis of the reservoir. In other words, the peripheral part of the membrane is sandwiched between the securing ring and the collar of the reservoir. There is therefore contact, preferably direct contact, between the membrane and the securing ring and between the membrane and the collar of the reservoir. This is a particularly simple version of the dispensing device, especially if the membrane is sandwiched directly between the securing ring and the collar of the reservoir, with no intermediate part.
The securing ring is fixed directly around the collar of the reservoir, preferably crimped around the collar of the reservoir. Thus, it is particularly simple to fix the membrane.
The product is a liquid product, preferably intended to be introduced into a subject. For example, the product is a medication.
The membrane is made of elastomer material, preferably bromobutyl-based or chlorobutyl-based. Thus, the membrane can be produced in a simple and inexpensive way and the membrane has no chemical effect on a product contained in the reservoir. In addition, these materials are advantageous in terms of sealing and their compatibility with medication products as well as with sterilization processes.
The dispensing device comprises the needle as needle for connecting to a product distribution tubing, the needle being configured to be distant from the membrane when the membrane is in the first stable position and to pass through the membrane when the membrane is in the second stable position. Thus, if the membrane switches into its second stable position when product starts to be dispensed, the product is only in contact with the needle and the distribution tubing when the product is dispensed. The sterility of the product in the reservoir is thus preserved until the product is dispensed out of the reservoir.
The needle is configured to allow product to be dispensed out of the reservoir via the needle when it passes through the membrane in the second stable position. Thus, it is particularly simple to dispense the product.
The needle is fixedly mounted with respect to the reservoir.
The needle is fixedly mounted on the reservoir. Thus, it is particularly easy to position the needle with respect to the collar of the reservoir without the need for a complex assembly of parts, such that the membrane is pierced simply and reliably, when the membrane changes to the second stable position. It is understood that the needle is preferably mounted indirectly on the reservoir.
The dispensing device comprises a piercing member comprising:
   a needle receiving element, to which the needle is connected, the needle advantageously being inserted in said needle receiving element and,
   a fixing element on the securing ring and/or on the collar of the reservoir. Thus, it is very simple to fix the needle receiving element and therefore the needle on the reservoir, while ensuring simple and reliable positioning of the needle with respect to the collar of the reservoir.
The piercing member is fixed to the reservoir such that an assembly sealed against ambient air is obtained between the needle and the membrane. Thus, the sterility of the product contained in the reservoir is further preserved.
The needle receiving element is fixed above the securing ring such that the securing ring comprises at least one portion positioned between the needle receiving element and the membrane along a central axis of the reservoir.
The needle receiving element with the membrane can form a chamber sealed against ambient air when the membrane bulges out in the first direction.
The fixing element and/or the needle receiving element are made of thermoplastic material. Thus, the piercing member is simple and inexpensive to produce.
The piercing member comprises a seal resting against the securing ring. Advantageously, the seal is positioned between the needle receiving element and the securing ring and rests against the securing ring in an axial direction.
The fixing element comprises clipping tabs clipped onto the securing ring and/or the collar of the reservoir. Thus, it is particularly simple to fix the needle to the reservoir. In addition, once assembled, intended or accidental dismantling of the needle from the reservoir is avoided or made impossible without damaging the various assembled elements. Correct operation of the assembly formed by the membrane and the needle is therefore ensured.

As an alternative, the fixing element has the shape of a clamping collar, which comprises a clipping tab clipped in a ring. Thus, it is particularly simple to fix the needle to the reservoir. In addition, once assembled, intended or accidental dismantling of the needle from the reservoir is avoided. Correct operation of the assembly formed by the membrane and the needle is therefore ensured. Furthermore, clipping is carried out tangentially, which avoids excessive compression that could deform the securing ring. The positioning of the needle with respect to the reservoir, and consequently of the needle with respect to the membrane, are therefore more precise.
The needle receiving element is integral with the fixing element.
As an alternative, the needle receiving element can be assembled with the fixing element.
The needle receiving element and the fixing element comprise additional means for assembling the needle receiving element to the fixing element. Advantageously, the needle receiving element comprises a circumferential element, such as a groove or rib, that can cooperate with a corresponding circumferential element, such as a rib or groove, of the fixing element.
The needle receiving element comprises, along a central axis of the reservoir, a first end passed by the needle and with respect to which a piercing end of the needle protrudes and a second end, opposite to the first end, covered by a protective element such as a film or cap isolating it from an outside environment.
The needle receiving element comprises a channel for dispensing a product, opened on its second end, and extending between the second end and the needle.
The needle extends through the needle receiving element and comprises a first end for piercing the membrane and a second end that can be connected directly or indirectly to a product distribution tubing.
The needle and the dispensing device are assembled in a housing such that the needle is contained in a piercing member comprising one or more fixing elements intended to interact with the housing, so that the needle is fixed with respect to the reservoir. Thus, the assembly remains simple but with no stress or direct assembly link between the needle and the reservoir or the securing ring, which simplifies their production.
The maximum distance between the first stable position and the second stable position is between 3 mm and 10 mm, preferably equal to 5 mm. Thus, the space required for the membrane to operate is compact. The “maximum distance between the first stable position and the second stable position” means the maximum displacement between the two positions of the membrane. If the bistable membrane has the general shape of a bulged disc, the “maximum distance between the first stable position and the second stable position” is defined as the distance travelled by the center of the membrane between the first stable position and the second stable position.
The end of the needle closest to the membrane is axially distant from the membrane when the membrane is in the first stable position, by a value less than or equal to the difference between the maximum distance between the first stable position and the second stable position and the thickness of the membrane, this value being from 2 mm to 7 mm, preferably less than 4 mm. This therefore ensures that the needle pierces the membrane in the second stable position.
The membrane comprises a central part that is deformable between the first stable position and the second stable position, the thickness of the central part being between 0.3 mm and 2.5 mm, preferably equal to 1 mm.
The thickness of the peripheral part is between 1 and 5 times the thickness of the central part, preferably between 2 and 4 times the thickness of the central part. Thus, it is easier to fix the membrane using the securing ring.
The membrane has the shape of a disc and its diameter is between 7 mm and 20 mm, preferably equal to 13 mm. The membrane is therefore compact.
The central part has the shape of a disc and its diameter is between 5 mm and 16 mm, preferably equal to 9 mm. The membrane therefore has a small area of contact with the product contained in the reservoir, which improves the sterility of the product contained in the reservoir.
The membrane is configured to switch from a first stable position into a second stable position when the differential pressure between the inner side and the outer side of the membrane is greater than a predetermined threshold. Thus, switching from the first stable position into the second stable position can be carried out simply by varying the pressure applied to the membrane.
The predetermined threshold is between 0.4 bar and 3 bar, preferably between 0.6 bar and 0.7 bar. Thus, the differential pressure required to switch the membrane from the first stable position into the second stable position is relatively low, and advantageously lower than the differential pressure which would be required to inflate a membrane in order to burst it. Thus, the membrane is pierced without the need to apply high pressure, which is particularly advantageous when energy must be saved or when the reservoir is designed to withstand low pressure.
The dispensing device comprises a pump and an electric motor to drive the pump, the pump being configured to switch the membrane from the first stable position into the second stable position and to dispense product out of the reservoir via the needle when the membrane is in the second stable position. Thus, operation of the membrane is particularly simple, since no additional actuation elements other than those required for dispensing are required.
The dispensing device comprises a battery to power the electric motor.
The pump is a pump configured to compress the product contained in the reservoir, such as for example a pump driving a piston sliding in the reservoir. Thus, the pump is particularly simple to produce. As an alternative, the pump is a peristaltic pump acting on the distribution tubing connected to the connection needle. Thus, the pump can be used to suck product out of the reservoir. Thus, the pump is particularly simple to produce.
The pump is configured so as to produce a differential pressure that is at least equal to the predetermined threshold between the inner side and the outer side of the membrane.
The dispensing device comprises a system for determining the position of the membrane. Thus, correct operation of the membrane can be checked, for example by checking that the membrane has not accidentally taken its second stable position before dispensing is required.
The system for determining the position of the membrane comprises a member for measuring the current or torque of the electric motor driving the pump. Thus, a means for checking the operation of the membrane is obtained, that is particularly simple and reliable. When the membrane switches from the first stable position to the second stable position, the pressure varies considerably and quickly, which can be detected by the measuring member.
The system for determining the position of the membrane comprises a pressure sensor configured to measure a pressure on the inner side and/or the outer side of the membrane, preferably configured to measure a differential pressure between the inner side and the outer side of the membrane. Thus, a simple means for checking the pressure and consequently the operation of the membrane is obtained. When the membrane switches from the first stable position to the second stable position, the pressure varies considerably and quickly, which can be detected by the pressure sensor.
The system for determining the position of the membrane comprises a sensor for detecting the position of the membrane, preferably a sensor selected from the group comprising an optical sensor, an electromagnetic sensor, an inductive sensor, a capacitive sensor, an impact sensor, an ultrasonic sensor, a contact sensor. Thus, a simple and standard means for checking the position of the membrane is obtained.

The invention also relates in particular to a system for dispensing a product comprising a dispensing device for dispensing a product as described previously, and a product dispensing unit.

According to other optional characteristics of the dispensing system taken alone or in combination:
The product dispensing unit comprises an insertion device for inserting a needle to insert and inject a product at an adjustable depth to allow different types of injection.
The insertion device comprises a needle housing on which the insertion needle is mounted, the needle housing being movably mounted between various positions including at least one insertion position.
The insertion device comprises a catheter movably mounted with respect to the needle and a catheter holder adapted to move the catheter with the needle when the needle housing moves to the at least one insertion position, and to separate it from the needle so that the catheter movement remains locked when the needle housing retracts from the at least one insertion position.
The insertion device comprises an insertion needle, which is different from the needle included in the dispensing device, and which is used to make an insertion in a site.
The dispensing system is portable. This allows the user to move and perform simple activities while receiving the product which may be, for example, a medical product in the context of a therapeutic treatment.

The invention also relates in particular to an assembly kit for a system for dispensing a product, comprising a dispensing device as described previously, and preferably a product dispensing unit.

According to other optional characteristics of the assembly kit taken alone or in combination:
The assembly kit comprises a first module comprising the dispensing device for dispensing product and a product dispensing unit, and a second module comprising means for adjusting the product dispensing unit and, optionally, means for checking the product dispensing unit.
The first module is removably mounted on the second module.
The adjustment means and/or the checking means are connected to the dispensing device for dispensing product, preferably interacting with the system for determining the position of the membrane.
The adjustment means comprise a wheel or a screw-nut assembly that can be accessed by a user.
The adjustment means comprise electronic control means.
The control means comprise at least one distance sensor and/or at least one displacement sensor.
The at least one distance sensor and/or the at least one displacement sensor can be inductive, capacitive, based on optics, ultrasounds or microwaves, or optoelectronic.
The assembly kit comprises a pump configured to switch the membrane from the first stable position into the second stable position and to dispense product out of the reservoir via the connecting needle when the membrane is in the second stable position.
The pump is directly integrated in the first module or in the second module.
A motor can be integrated in the first module or the second module. The motor is used to drive the pump according to one embodiment. Similarly, optionally, the motor can be used to actuate and/or drive the product dispensing unit. Preferably, the motor is an electric motor.
The first module of the assembly kit corresponds to a disposable part, and the second module corresponds to a reusable part.

Thus, an inexpensive dispensing system is obtained, in which the expensive and reusable elements, for example a motor, are integrated in a reusable part, while preserving safety, since the elements in contact with the product, for example the dispensing device, are not reusable and are grouped in the disposable part, quite separate from the reusable part.

The assembly kit allows, for example, the medical staff or the patient to have everything needed to perform a treatment or to provide the medical staff or the patient with some of the items required by the system for dispensing a product into a site, in particular one or more disposable parts.

### BRIEF DESCRIPTION OF THE DRAWINGS

The invention will be better understood on reading the following description, given solely by way of example and with reference to the accompanying drawings in which:

FIG. 1 is a schematic cross-sectional view of a dispensing device according to a first embodiment.

FIG. 2 is a schematic cross-sectional view of a detail of the dispensing device according to the first embodiment, in which the membrane is in its first stable position.

FIG. 3 is a schematic cross-sectional view of a detail of the dispensing device according to the first embodiment, in which the membrane is in its second stable position.

FIG. 4 is a schematic cross-sectional perspective view of a detail of a dispensing device according to a second embodiment.

FIG. 5 is a schematic perspective view of a detail of the dispensing device according to the second embodiment.

### DETAILED DESCRIPTION OF THE INVENTION

On all the figures, the same references refer to the same elements. The following embodiments are examples. Although the description refers to one or more embodiments, this does not necessarily mean that each reference concerns the same embodiment, or that the characteristics apply only to a single embodiment. Simple characteristics of different embodiments may also be combined to provide other embodiments.

FIG. 1 shows a dispensing device according to a first embodiment, designated by the general reference 1.

The dispensing device 1 for dispensing product comprises a reservoir 3 of product P, a membrane 5, a securing ring 7 and a needle 8. The dispensing device 1 is portable.

The reservoir 3 is provided with a collar 9 delimiting an opening 11. More precisely in this example, the reservoir 3 has the shape of a syringe provided with the collar 9. The reservoir 3 is made of glass or thermoplastic material. In this example, the product P contained in the reservoir 3 is a liquid product, preferably intended to be introduced into a subject, for example a human patient. For example, the product P is a medication. In a variant, not shown, the reservoir has the shape of a cartridge provided with a collar. In another variant, not shown, the reservoir has a flexible pouch connected to a collar that is more rigid than the flexible pouch, and that is used to fix the membrane via the securing ring 7 to the collar.

The products, in particular pharmaceutical products, likely to be used by the dispensing device include for example formulations containing at least one active ingredient such as peptides, proteins, hormones, active ingredients of biological origin, nucleotide-based active ingredients, such as for example DNAs, RNAs or oligonucleotides, active ingredients of molecular weight up to 1500 Da, polysaccharides, vaccines, enzymes, antibodies, nutritional formulae and other substances or a mixture thereof.

The products, in particular pharmaceutical products, likely to be used by the dispensing device can be used for the treatment and/or prevention of diabetes, thromboses, cardiovascular diseases, such as coronary syndrome, angina, myocardial infarction, cancers, macular degeneration, inflammations, atheroscleroses and/or rheumatoid arthritis.

These active ingredients may include, but are not limited to, insulins, insulin analogues such as insulin lispro or insulin glargine, insulin derivatives, C-peptide, GLP-1 receptor agonists such as dulaglutide or liraglutide, glucagon, glucagon analogues, glucagon derivatives, gastric inhibitor polypeptides (GIP), GIP analogues, GIP derivatives, oxyntomodulin analogues, oxyntomodulin derivatives, therapeutic antibodies, such as monoclonal antibodies and any therapeutic agent that can be delivered by the above device, as well as a pharmaceutically acceptable salt and/or solvate and/or hydrate of the active ingredients listed above. The medication as it is used in the device can be formulated with one or more excipients.

In particular, the product may comprise at least one peptide for the treatment and/or prevention of diabetes.

In particular, the product may comprise at least one human insulin or an insulin derivative or analogue such as for example glucagon-like peptide (GLP-1) or a GLP-1 analogue or derivative, or exedin-3, exedin-4 or one of their analogues or derivatives, or Gly(A21), Arg(B31), Arg(B32) human insulins; Lys(B3), Glu(B29) human insulins; Lys(B28), Pro(B29) human insulins, Asp(B28) human insulins, B29-N-myristoyl-des(B30) human insulins; B29-N-palmitoyl-des(B30) human insulins; B29-N-myristoyl human insulins; B29-N-palmitoyl human insulins; B28-N-myristoyl LysB28ProB29 human insulins; B28-N-palmitoyl-LysB28ProB29 human insulins; B30-N-myristoyl-ThrB29LysB30 human insulins; B30-N-palmitoyl-ThrB29LysB30 human insulins; B29-N—(N-palmitoyl-Y-glutamyl)-des(B30) human insulins; B29-N—(N-lithocholyl-Y-glutamyl)-des(B30) human insulins; B29-N-(ω-carboxyheptadecanoyl)-des(B30) human insulins and B29-N-(ω-carboxyheptadecanoyl) human insulins and/or a pharmaceutically acceptable salt and/or solvate and/or hydrate of the insulins listed above.

In particular the product may comprise at least one hormone such as for example the hypophyseal or hypothalamic hormones such as for example the gonadotropins (follitropins, lutropins, choriongonadotropins, menotropins), somatropins or somatotropins, desmopressins, terlipressins, gonadorelins, triptorelins, leuprorelins, buserelins, nafarelins, goserelins and/or a pharmaceutically acceptable salt and/or solvate and/or hydrate of the hormones listed above.

In particular the product may comprise at least one polysaccharide such as for example a glucosaminoglycan, a hyaluronic acid, a heparin, a low-molecular-weight heparin or a heparin derivative, a sulphated or polysulphated polysaccharide, and/or a pharmaceutically acceptable salt and/or solvate and/or hydrate of the polysaccharides listed above.

The pharmaceutically acceptable salts are for example the acid addition salts, such as for example the HCl or HBr salts, and the basic salts, such as for example the salts composed of an alkali cation such as Na+, K+, or Ca2+, or an ammonium ion of type N+(R1)(R2)(R3)(R4), where R1, R2, R3 and R4 represent independently of each other: a hydrogen atom, an optionally substituted alkyl group (C1-C6), an optionally substituted alkenyl group (C2-C6), an optionally substituted aryl group (C6-C10), or an optionally substituted heteroaryl group (C6-C10). Other examples of pharmaceutically acceptable salts are well known by those skilled in the art.

The securing ring 7 is used for fixing the membrane 5 to the collar 9. In this example, the securing ring 7 is fixed directly around the collar 9 of the reservoir 3. More precisely, the securing ring 7 is crimped around the collar 9 of the reservoir 3 and has a diameter that is smaller at its end distant from the membrane so as to cooperate with the collar 9 and thus prevent it from being extracted out of the collar 9. The securing ring 7 is made of thermoplastic material or metallic material, preferably aluminum.

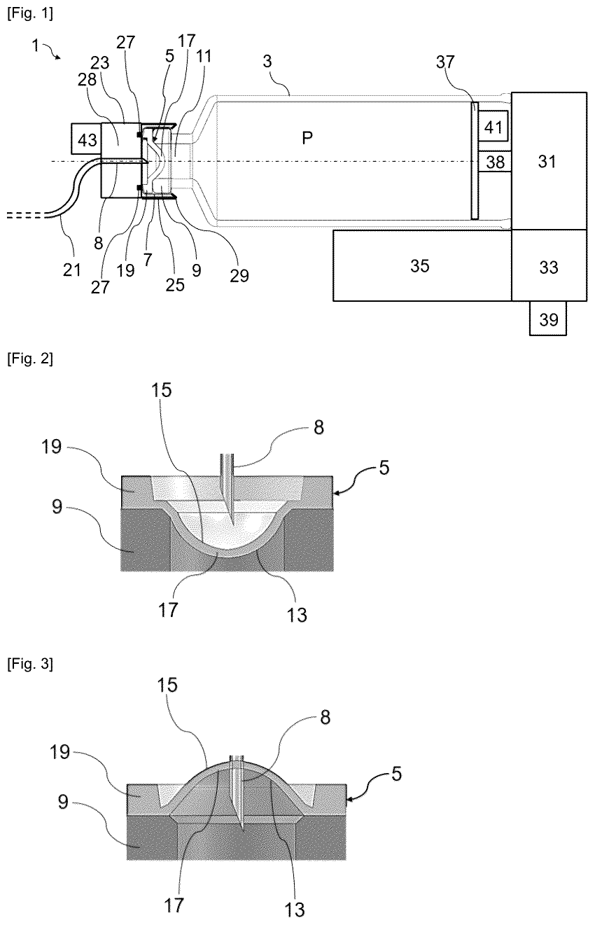

The membrane 5 closes the opening 11 and thus has an inner side 13 oriented towards the reservoir 3 and an outer side 15 away from the inner side 13. The membrane 5 is a bistable membrane. Thus, the membrane 5 is configured to switch from a first stable position shown in detail on FIG. 2, in which the membrane 5 bulges out in a first direction such that the membrane 5 protrudes into the reservoir 3 and closes the reservoir 3, into a second stable position shown in detail on FIG. 3, in which the membrane 5 bulges out in a second direction opposite to the first direction. More precisely, the membrane 5 switches from the first stable position into the second stable position when the differential pressure between the inner side 13 and the outer side 15 of the membrane 5 is greater than a predetermined threshold. The predetermined threshold is between 0.4 bar and 3 bar, preferably between 0.6 bar and 0.7 bar. Thus, in this example, in its first stable position, the membrane 5 closes the reservoir 3 so as to prevent product P from being dispensed, and in its second stable position, the membrane 5 bulges out in the second direction such that it protrudes out of the reservoir 3. In this example, the opening 11 is closed only by the membrane 5. The membrane 5 is mounted directly against the collar 9 of the reservoir 3, the membrane 5 being crimped directly on the collar 9 of the reservoir 3 by the securing ring 7. The membrane 5 has the shape of a disc and its diameter is between 7 mm and 20 mm, preferably equal to 13 mm. As shown on FIG. 2, during assembly and storage, the membrane 5 bulges out in the first direction such that it protrudes into the collar 9 of the reservoir 3, through the opening 11. The membrane 5 comprises a peripheral part 19 and a central part 17 that is deformable between the first stable position and the second stable position. The thickness of the central part 17 is between 0.3 mm and 2.5 mm, preferably equal to 1 mm. The central part 17 has the shape of a disc and its diameter is between 5 mm and 16 mm, preferably equal to 9 mm. The peripheral part 19 is fixed since it is compressed axially between the securing ring 7 and the collar 9 of the reservoir 3. In a non-compressed state, the thickness of the peripheral part 19 is between 1 and 5 times the thickness of the central part 17, preferably between 3 and 4 times the thickness of the central part 17. The maximum distance between the first stable position and the second stable position is between 3 mm and 10 mm, preferably equal to 5 mm. This maximum distance corresponds to the distance between the center of the membrane 5 located on the central part 17 bulging out in the first direction in the first stable position and the center of the membrane 5 located on the central part 17 bulging out in the second direction in the second stable position. The membrane 5 is made of elastomer material, preferably bromobutyl-based or chlorobutyl-based. When the membrane 5 switches into its second stable position, it bulges out in the second direction so as to be pierced by the needle 8 disposed on the outer side 15 of the membrane 5.

In this example, the needle 8 is a needle for connecting to a product P distribution tubing 21, preferably a needle for connecting to a catheter for dispensing product. The needle 8 is made of metallic material, for example stainless steel. According to a variant, the needle 8 is made of plastic material which is preferably thermoplastic. For example, the needle 8 is connected to an insertion device via the distribution tubing 21. This insertion device comprises an insertion needle used to insert into a site the product P contained in the reservoir 3 and dispensed via the needle 8 when the membrane 5 is in its second stable position. Advantageously, this insertion device may also comprise a needle housing on which the insertion needle is mounted, the needle housing being movably mounted between various positions including at least one insertion position. Preferably, the insertion device comprises a catheter movably mounted with respect to the insertion needle and a catheter holder adapted to move the catheter with the insertion needle when the needle housing moves to the at least one insertion position, and to separate it from the insertion needle so that the catheter movement remains locked when the needle housing retracts from the at least one insertion position.

According to a variant, not shown, the needle is a needle for injecting product, which comprises one end for piercing the membrane 5 and one opposite end for injecting product into a site of a subject.

The needle 8 is distant from the membrane 5 when the membrane 5 is in the first stable position and passes through the membrane 5 when the membrane 5 is in the second stable position. Thus, the needle 8 can be used to dispense the product P out of the reservoir 3 via the needle 8 when it passes through the membrane 5 in the second stable position. The needle 8 is fixedly mounted with respect to the reservoir 3. In this example, the needle 8 is fixedly mounted on the reservoir 3. To do this, the needle 8 is inserted in a piercing member 23, which comprises a fixing element 25 on the securing ring 7 and/or on the collar 9 of the reservoir 3. The piercing member 23 is also called a fixing member and comprises the fixing element 25 and a needle 8 receiving element 28 in which the needle 8 is connected, advantageously fitted inside. The needle 8 receiving element 28 is fixed above the securing ring 7 such that the securing ring 7 comprises at least one portion positioned between the needle 8 receiving element 28 and the membrane 5 along a central axis of the reservoir 3. The needle 8 extends through the needle 8 receiving element 28 and comprises a first end for piercing the membrane 5 and a second end that can be connected directly or indirectly to the product P distribution tubing 21.

The fixing element 25 and/or the needle 8 receiving element 28 are made of thermoplastic material. In this example, the piercing member 23 is made of thermoplastic material. The piercing member 23 is fixed to the reservoir 3 such that an assembly sealed against ambient air is obtained between the needle 8 and the membrane 5. Thus, the needle 8 receiving element 28 with the membrane 5 can form a chamber sealed against ambient air, when the membrane bulges out in the first direction. To do this, the piercing member 23 comprises a seal 27 resting against the securing ring 7. In this example, the seal 27 is positioned between the needle 8 receiving element 28 and the securing ring 7 and rests against the securing ring 7 in an axial direction.

The needle receiving element 28 comprises, along a central axis of the reservoir, a first end passed by the needle 8 and with respect to which a piercing end of the needle 8 protrudes and a second end, opposite to the first end. The needle receiving element 28 comprises a channel for dispensing product P opening out onto its second end and extending between the second end and the needle 8. According to a variant, the second end of the needle receiving element 28 is covered by a protective element such as a film or cap isolating it from an outside environment. The protective element is removed before connecting directly or indirectly the second end of the needle 8 to the product P distribution tubing 21.

In this example, the seal 27 is annular. The fixing element 25 comprises clipping tabs 29 clipped onto the securing ring 7, around the collar 9, and/or onto the collar 9 of the reservoir 3. In this example, the fixing element 25 is made in one piece with the piercing member 23. In other words, the needle 8 receiving element is integral with the fixing element. As an alternative, according to a variant, the needle 8 receiving element can be assembled with the fixing element. According to this variant, the needle 8 receiving element and the fixing element comprise additional means for assembling the needle 8 receiving element to the fixing element. Advantageously, according to this variant, the needle 8 receiving element comprises a circumferential element, such as a groove or a rib, that can cooperate with a corresponding circumferential element, such as a rib or a groove, of the fixing element.

The needle 8 is disposed so as to pass through the membrane 5 when the membrane 5 is in the second stable position. Thus, the dispensing device 1 comprises the needle 8 that can pierce the membrane 5 when the membrane 5 is in the second stable position. To do this, the end of the needle 8 closest to the membrane 5 is axially distant from the membrane 5, more precisely with respect to the outer side 15 of the membrane 5, when the membrane 5 is in the first stable position. The value of this distance is less than or equal to the difference between the maximum distance between the first stable position and the second stable position and the thickness of the membrane 5, this value being between 2 and 7 mm, preferably less than 4 mm.

To dispense product P, the dispensing device 1 comprises in this example a pump 31, an electric motor 33 driving the pump 31 and a battery 35 to power the electric motor 33.

The pump 31 is configured to switch the membrane 5 from the first stable position into the second stable position and to dispense product out of the reservoir 3 via the needle 8 when the membrane 5 is in the second stable position. In this example, the pump 31 is a pump configured to compress the product P contained in the reservoir 3. For example, the pump 31 drives a piston 37 sliding in the reservoir 3. According to a second variant, not shown, the pump 31 is a peristaltic pump acting on the distribution tubing 21 connected to the needle 8.

The pump 31 is configured so as to produce a differential pressure that is at least equal to the predetermined threshold between the inner side 13 and the outer side 15 of the membrane 5. Thus, the pump 31 both allows the distribution tubing 21 to communicate with the reservoir 3 via the needle 8 when the membrane 5 switches from the first stable position into the second stable position, then the actual dispensing of the product P via the distribution tubing 21.

To check the operation of the membrane 5, the dispensing device 1 comprises a system for determining the position of the membrane 5.

As shown on FIG. 1, the system for determining the position of the membrane 5 comprises a member 39 for measuring the current or torque of the electric motor 33 driving the pump 31. The system for determining the position of the membrane also comprises a pressure sensor 41 configured to measure a pressure on the inner side 13 and/or the outer side 15 of the membrane 5, preferably configured to measure a differential pressure between the inner side 13 and the outer side 15 of the membrane 5. The system for determining the position of the membrane 5 further comprises a sensor 43 for detecting the position of the membrane 5, preferably a sensor selected from the group comprising an optical sensor, an electromagnetic sensor, an inductive sensor, a capacitive sensor, an impact sensor, an ultrasonic sensor, a contact sensor.

To assemble a dispensing system comprising the dispensing device 1, a first module is produced comprising the reservoir 3 containing product P, the securing ring 7, the membrane 5, the needle 8, a first part of the system for determining the position of the membrane 5 and a product dispensing unit (not shown). A second module is also produced comprising the pump 31 and the electric motor 33 for driving the pump 31, a second part of the system for determining the position of the membrane 5 as well as means (not shown) for adjusting the product dispensing unit, the pump 31 being configured to switch the membrane 5 from the first stable position into the second stable position and to dispense product P out of the reservoir 3 via the needle 8 when the membrane 5 is in the second stable position. The first module and the second module thus form an assembly kit, the first module being single-use and the second module being reusable in the configuration described. An assembly kit for a system for dispensing a product means that such an assembly kit comprises elements disposed separately, in this case the first module and the second module, which are intended to be assembled subsequently to form a system for dispensing product. Thus, the assembly kit for a system for dispensing a product comprises a dispensing device 1 and a product dispensing unit.

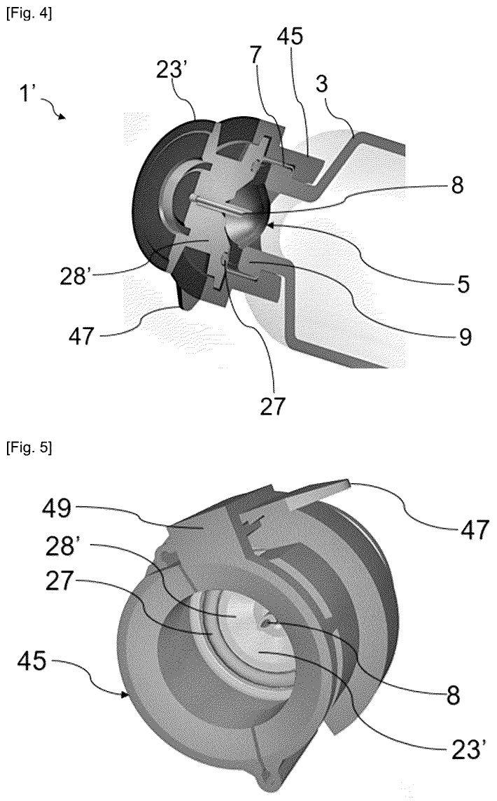

FIGS. 4 and 5 show a detail of a dispensing device 1′ according to a second embodiment. The dispensing device 1′ according to this second embodiment is different from the dispensing device 1 according to the first embodiment in that the piercing member 23′, also called fixing member, comprises a fixing element 45 that is structurally different from the fixing element 25 of the first embodiment, but performs the same function of fixing the needle 8 on the reservoir 3 to position it with respect to the membrane 5. In this second embodiment, the fixing element 45 has the shape of a clamping collar, which comprises a clipping tab 47 clipped in a ring 49. Thus, clipping is carried out tangentially. The fixing element 45 is for example made of thermoplastic material. During assembly, the piercing member 23′ is pre-positioned with respect to the reservoir 3, and the fixing element 45 is then attached and clamped both to the piercing member 23′ and to the securing ring 7 and/or to the collar 9 of the reservoir 3. Clamping is carried out by clipping the clipping part 47 in the ring 49. In this embodiment, the piercing member 23 also comprises an annular seal 27, resting against the securing ring 7 when the dispensing device 1′ is in the assembled state. In this example, the needle 8 receiving element 28′ can be assembled with the fixing element 45. Thus, the needle 8 receiving element 28′ and the fixing element 45 comprise additional means for assembling the needle receiving element 28′ to the fixing element 45. Advantageously, the needle receiving element 28′ comprises a circumferential element, such as a groove or a rib, that can cooperate with a corresponding circumferential element, such as a rib or a groove, of the fixing element 45.

The needle receiving element 28′ comprises along a central axis of the reservoir a first end passed by the needle 8 and with respect to which a piercing end of the needle 8 protrudes and a second end, opposite to the first end, covered by a protective element such as a film or cap isolating the second end from an outside environment. The needle 8 receiving element 28′ comprises a product P dispensing channel opened on its second end and extending between the second end and the needle 8.

In the embodiment shown on the figures, the needle is a bevel needle whose end for piercing the membrane comprises an opening oriented towards the membrane and through which the product can flow. When dispensing product, the product can then flow through the needle from the piercing end towards a dispensing end opposite to the piercing end along a longitudinal axis of the needle. As a variant, the needle is a pencil point needle comprising, between the membrane piercing end and a dispensing end opposite to the piercing end along a longitudinal axis of the needle, a side opening through which the product can flow. When dispensing product, the product can then flow through the needle from the side opening towards the dispensing end.

The invention is not limited to the embodiments described and other embodiments will be clearly apparent to those skilled in the art. In particular, the embodiments can be combined together.

### LIST OF REFERENCES

1, 1′: dispensing device
3: reservoir
5: membrane
7: securing ring
8: needle
9: collar
11: opening
13: inner side
15: outer side
17: central part
19: peripheral part
21: distribution tubing
23, 23′: piercing member
25: fixing element
27: seal
28, 28′: receiving element
29: clipping tab
31: pump
33: electric motor
35: battery
37: piston
38: rod
39: measuring member
41: pressure sensor
43: detection sensor
45: fixing element
47: clipping tab
49: ring
P: product

1. A dispensing device for dispensing a product, comprising:
a reservoir of configured to contain the product, wherein the reservoir is provided with a collar delimiting an opening,
a membrane closing the opening and having an inner side oriented towards the reservoir and an outer side opposite to the inner side,
a securing ring configured to secure the membrane to the collar,
the membrane being configured to switch from a first stable position, in which the membrane bulges out in a first direction such that it protrudes into the reservoir and closes the reservoir, into a second stable position, in which the membrane bulges out in a second direction opposite to the first direction so as to be pierced by a needle disposed on the outer side of the membrane.

2. The dispensing device according to claim 1, wherein the membrane is made of an elastomer material.

3. The dispensing device according to claim 1, wherein the needle is configured to connect the reservoir to a product distribution tubing and configured to be distant from the membrane when the membrane is in the first stable position and to pass through the membrane when the membrane is in the second stable position.

4. The dispensing device according to claim 1, wherein the needle is fixedly mounted with respect to the reservoir.

5. The dispensing device according to claim 1, wherein the membrane has a peripheral part compressed axially between the securing ring and the collar of the reservoir.

6. The dispensing device according to claim 1, wherein the membrane comprises a central part that is deformable between the first stable position and the second stable position, the central part having a thickness between 0.3 mm and 2.5 mm.

7. The dispensing device according to claim 1, wherein the membrane is configured to switch from the first stable position into the second stable position when a differential pressure between the inner side and the outer side of the membrane is greater than a predetermined threshold.

8. The dispensing device according to claim 7, wherein the predetermined threshold is between 0.4 bar and 3 bar.

9. The dispensing device according to claim 1, further comprising a pump and an electric motor to drive the pump, the pump being configured to switch the membrane from the first stable position into the second stable position and to dispense the product out of the reservoir via the needle when the membrane is in the second stable position.

10. The dispensing device according to claim 1, further comprising a system for determining a position of the membrane.

11. The dispensing device according to claim 10, wherein the system for determining the position of the membrane comprises a member for measuring a current or torque of the electric motor driving the pump.

12. The dispensing device according to claim 10, wherein the system for determining the position of the membrane comprises a pressure sensor configured to measure a pressure on the inner side and/or on the outer side of the membrane.

13. A system for dispensing a product comprising a dispensing device according to claim 1 and a product dispensing unit.

14. An assembly kit for a system for dispensing a product, comprising the dispensing device according to claim 1 and a product dispensing unit.

15. The dispensing device according to claim 2, wherein the membrane is made of bromobutyl-based material and/or chlorobutyl-based material.

16. The dispensing device according to claim 8, wherein the predetermined threshold is between 0.6 bar and 0.7 bar.

17. The dispensing device according to claim 12, wherein the pressure sensor is configured to a differential pressure between the inner side and the outer side of the membrane.
